(19) United States
(12) Patent Application Publication
(10) Pub. No.: US 2018/0296521 A1
(43) Pub. Date: Oct. 18, 2018

(54) METHODS AND COMPOSITIONS FOR TREATING GASTROINTESTINAL INFLAMMATION

(71) Applicant: SCYTHIAN BIOSCIENCES INC., Toronto (CA)

(72) Inventor: David A. Schrader, New York, NY (US)

(21) Appl. No.: 15/767,747

(22) PCT Filed: Oct. 17, 2016
(86) PCT No.: PCT/US2016/057313

§ 371 Date: Apr. 12, 2018

Related U.S. Application Data
(60) Provisional application No. 62/242,469, filed on Oct. 16, 2015.

(51) Int. Cl. A61K 31/352; A61K 45/06; A61K 31/05; A61K 31/164

(52) U.S. Cl. CPC A61K 31/352; A61K 31/164; A61K 31/05; A61K 45/06

(57) ABSTRACT

Methods and compositions for treating gastrointestinal inflammation in a subject are provided.

This patent application claims the benefit of priority from U.S. Provisional Patent Application Ser. No. 62/242,469, filed Oct. 16, 2015, the content of which is hereby incorporated by reference in its entirety.

### BACKGROUND

Gastrointestinal inflammation is one of the most common types of inflammatory process which affects humans. It is diverse in aetiology, pathogenesis and manifestation.

Some examples of gastrointestinal inflammation can include gastritis, esophagitis, and colitis, all named for different parts of the digestive tract. In the involved area, large numbers of white blood cells are present to counter a perceived threat to the body. They can cause swelling, redness, tenderness, and irritation, while extreme inflammation can form lesions which may bleed. Patients with gastrointestinal inflammation may notice symptoms like mucus and blood in the stool, trouble swallowing, loss of appetite, and abdominal discomfort.

In many individuals, gastrointestinal inflammation is an immune response in the digestive tract that can cause symptoms including nausea, cramping, and diarrhea. Physician's often use a specific diagnostic term like colitis, referring to inflammation in the bowel, to discuss a patient's case. There are a number of reasons for patients to experience this common medical problem and testing can explore some possible explanations and help the medical provider develop some treatment recommendations to help the patient resolve the issue. These may involve an assortment of options including medications, lifestyle modifications, and complementary medicine like acupuncture or massage.

Treatments have traditionally focused on altering diet, reducing gas, intestinal antispasmodics and intestinal lubrication. Steroidal and surgical treatments have been utilized for severe cases.

In recent years, a non-steroidal drug, Humira has become an approved treatment for these inflammatory conditions. Humira acts as a Tumor Necrosis Factor blocker. Humira must be administered as a shot and is an expensive drug, especially when used regularly.

Cannabidiol (CBD), a naturally occurring chemical in certain varieties of marijuana, acts as a CB-2 agonist and presents a broad range of anti-inflammatory and immune inhibitory effects. See for example http: with the extension theroc.us/images/CANNABINOID-BASED%20DRUGS%20AS%20ANTI-INFLAMMATORY%20THERAPEUTICS.pdf of the world wide web. By way of example, a synthetic CBD is currently undergoing testing for treatment of Lennox-Gestaut syndrome. See http: with the extension www.gwpharm.com/GW%20Pharmaceuticals%20Initiates%20Second%20Phase%203%20Pivotal%20Study%20of%20Epidiolex%20CBD%20in%20Lennox-Gastaut%20Syndrome.aspx of the world wide web. Research on the use of CBD and other cannabinoids as a possible treatment for various inflammatory disorders has been ongoing for no less than 25 years. A basic overview of some of the research conducted from 1982 to 2011 is provided at http:// with the extension www.calgarycmmc.com/antiinflammatory.htm of the world wide web.

### SUMMARY

The present invention relates to methods and compositions for treating gastrointestinal inflammation and/or immune responses associated with gastrointestinal inflammation in a subject.

An aspect of the present invention relates to a method for treating gastrointestinal inflammation in a subject suffering therefrom which comprises administering to the subject a first composition comprising a CB2 agonist, an agent which effectively increases an endogenous CB2 agonist and/or an agent which modifies levels of anandamide (AEA) or 2-arachidonoyl glycerol (2-AG). This method further comprises administering a second composition comprising a cannabinoid.

Another aspect of the present invention relates to a method for treating gastrointestinal inflammation in a subject suffering therefrom which comprises administering to the subject a first composition comprising a CB2 agonist, an agent which effectively increases an endogenous CB2 agonist and/or an agent which modifies levels of anandamide (AEA) or 2-arachidonoyl glycerol (2-AG). This method further comprises administering a second composition comprising an N-Methyl-D-aspartate (NMDA) receptor antagonist.

Another aspect of the present of the present invention relates to a method for treating gastrointestinal inflammation in a subject suffering therefrom wherein comprises administering to the subject a first composition comprising a CB2 agonist, an agent which effectively increases an endogenous CB2 agonist and/or an agent which modifies levels of anandamide (AEA) or 2-arachidonoyl glycerol (2-AG) and a second composition comprising a distinct CB2 agonist, agent which effectively increases an endogenous CB2 agonist and/or agent which modifies levels of anandamide (AEA) or 2-arachidonoyl glycerol (2-AG).

Another aspect of the present invention relates to a method for treating gastrointestinal inflammation in a subject suffering therefrom which comprises administering to the subject a first composition comprising cannabichromene.

Yet another aspect of the present invention relates to compositions for treatment of gastrointestinal inflammation. In one nonlimiting embodiment, the composition comprises a CB2 agonist and an agent which effectively increases an endogenous CB2 agonist or an agent which modifies levels of anandamide (AEA) or 2-arachidonoyl glycerol (2-AG). In one nonlimiting embodiment, the composition comprises a CB2 agonist and an inhibitor of fatty acid amide hydrolase (FAAH inhibitor). In another nonlimiting embodiment, the composition comprises a CB2 agonist, an agent which effectively increases an endogenous CB2 agonist or an agent which modifies levels of anandamide (AEA) or 2-arachidonoyl glycerol (2-AG), and an NMDA receptor antagonist. In one nonlimiting embodiment, the composition comprises a CB2 agonist, a FAAH inhibitor and an NMDA receptor antagonist.

In one nonlimiting embodiment, the subject is suffering from gastritis, esophagitis, colitis, inflammatory bowel disease, Crohn's disease, ulcerative colitis, an immune disorder characterized by gastrointestinal inflammation, or irritable bowel syndrome.

### DETAILED DESCRIPTION

The present invention provides methods and compositions for treating gastrointestinal inflammation in a subject. By gastrointestinal inflammation it is meant to be inclusive of gastritis, esophagitis, and/or colitis. In one nonlimiting embodiment, the subject is suffering from gastritis, esophagitis, colitis, inflammatory bowel disease, Crohn's disease, ulcerative colitis, an immune disorder characterized by gastrointestinal inflammation, or irritable bowel syndrome.

By “treating” or “treatment” or “treat” as used herein it is meant to include controlling, reducing or ameliorating inflammation and/or controlling, reducing or ameliorating any immune response associated with the gastrointestinal condition.

In one nonlimiting embodiment, the method of the present invention comprises administering to a subject suffering from gastrointestinal inflammation a first composition comprising a CB2 agonist, an agent which effectively increases an endogenous CB2 agonist and/or an agent which modifies levels of anandamide (AEA) or 2-arachidonoyl glycerol (2-AG).

By “CB2 agonist” as used herein, it is meant to include classes of agents which activate the cannabinoid 2 receptor in a selective or nonselective manner. In one nonlimiting embodiment, the CB2 agonist is a non-cannabinoid CB2 agonist. In another nonlimiting embodiment, the CB2 agonist is a cannabinoid CB2 agonist.

Alternatively, or in addition, the first composition may comprise an agent which effectively increases an endogenous CB2 agonist and/or an agent which modifies levels of anandamide (AEA) or 2-arachidonoyl glycerol (2-AG).

In one nonlimiting embodiment, the first composition comprises an agent which increases levels of AEA.

In another nonlimiting embodiment, the first composition comprises an agent which decreases levels of 2-AG.

In one nonlimiting embodiment, the first composition comprises an inhibitor of fatty acid amide hydrolase (FAAH inhibitor). FAAH inhibitors reduce the level of 2-AG through upregulation of anandamide levels without acting on the CB2 receptor. See Tourteau et al. Bioorg Med Chem Lett. 2014 Mar. 1; 24(5):1322-6. doi: 10.1016/j.bmc1.2014.01.056. Epub 2014 Jan. 28). Thus, the use of a FAAH inhibitor in accordance with the present invention would create a similar anti-inflammatory effect through an alternate mechanism without binding or affecting the CB2 receptor. Various FAAH inhibitors, including synthetic versions have been described. See Otrubova et al. Bioorg Med Chem Lett. 2011 Aug. 15; 21 (16): 4674-4685.

doi:10.1016/j.bmc1.2011.06.096).

In one nonlimiting embodiment, the first composition comprises AEA.

In one nonlimiting embodiment, the first composition is administered via a regimen effective to control, reduce or ameliorate inflammation and/or control, reduce or ameliorate any immune response associated with the gastrointestinal condition and/or alleviate symptoms associated with gastrointestinal inflammation in the subject.

In one nonlimiting embodiment, the first composition is administered daily or every other day until symptoms of the gastrointestinal inflammation are alleviated.

The first composition may be administered by any route providing for delivery of effective amounts of the CB2 agonist, the agent which effectively increases an endogenous CB2 agonist and/or the agent which modifies levels of anandamide (AEA) or 2-arachidonoyl glycerol (2-AG) to a portion of the gastrointestinal tract requiring treatment. Examples of routes of administration include, but are in no way limited to, intravenous, intranasal, oral, topical, transdermal or via inhalation.

In one nonlimiting embodiment, a CB-2 agonist and a FAAH inhibitor are both administered to the subject. In this embodiment, the agents can be administered simultaneously or at different times. In one nonlimiting embodiment, a composition comprising a CB-2 agonist and a composition comprising a FAAH inhibitor are combined into a single pharmaceutical formulation for simultaneous administration.

As will be understood by the skilled artisan upon reading this disclosure, dosages can be determined by the attending physician, according to the severity and/or extent of the gastrointestinal inflammation to be treated, method of administration, patient's age, weight, contraindications and the like.

In one nonlimiting embodiment of the present invention, the method further comprises administering to the subject a second composition comprising a cannabinoid.

By “cannabinoids”, as used herein, it is meant the class of agents including endocannabinoids, phytocannabinoids and those agents which are neither endocannabinoids or phytocannabinoids, and are referred to herein as “syntho-cannabinoids”. By the term “endocannabinoids” it is meant endogenous cannabinoids which are high affinity ligands of CB1 and CB2 receptors. By “phytocannabinoids” it is meant cannabinoids that originate in nature and can be found in the cannabis plant. Phytocannabinoids can be present in an extract including a botanical drug substance, isolated, or reproduced synthetically. By “syntho-cannabinoids” it is meant those compounds capable of interacting with the cannabinoid receptors (CB1 and/or CB2) which are not found endogenously or in the cannabis plant.

Nonlimiting examples of cannabinoids useful in the second composition include cannabidiol, tetrahydrocannabinol, cannabichromene, cannabinol, cannabigerol tetrahydrocannabivarin and delta-8-tetrahydrocannabinol.

In another nonlimiting embodiment of the present invention, the method further comprises administering to the subject a second composition comprising an N-Methyl-D-aspartate (NMDA) receptor antagonist.

By “NMDA receptor antagonist” as used herein, it is meant to include the class of agents that work to antagonize or inhibit the action of N-Methyl-D-aspartate receptor (NMDA). Examples include, but are not limited to, dizocilpine (MK-801), ketamine, memantine, phencyclidine, gascyclidine, AP5, amantadine, ibogaine, nitrous oxide riluzole, dextrophan, AP-7, tiletamine, midafotel, aptiganel and 7-hydroxy-delta6-tetrahydrocannabinol 1,1-dimethylheptyl (dexanabinol: HU-211). In one nonlimiting embodiment, the NMDA receptor antagonist is a noncompetitive NMDA receptor antagonist such as dexanabinol, GK-11 or gascyclidine, or phencyclidine or an uncompetitive NMDA receptor antagonist such as dizocilpine. Additional nonlimiting examples of NMDA receptor antagonists useful in the present invention are disclosed in U.S. Pat. No. 5,521,215, teachings of which are incorporated herein by reference in their entirety. In one nonlimiting embodiment, the NMDA receptor antagonist is 7-hydroxy-delta6-tetrahydrocannabinol 1,1-dimethylheptyl (Dexanabinol: HU-211).

In another nonlimiting embodiment of the present invention, the method further comprises administering to the subject a second composition comprising a CB2 agonist, an agent which effectively increases an endogenous CB2 agonist and/or an agent which modifies levels of anandamide (AEA) or 2-arachidonoyl glycerol (2-AG) that is distinct from that used in the first composition. CB2 agonists, agents which effectively increase an endogenous CB2 agonist and agents which modify levels of anandamide (AEA) or 2-arachidonoyl glycerol (2-AG) useful in the second composition are described supra.

In one nonlimiting embodiment, the second composition is administered via a regimen effective to control, reduce or ameliorate inflammation and/or control, reduce or ameliorate any immune response associated with the gastrointestinal condition and/or alleviate symptoms associated with gastrointestinal inflammation in the subject.

In one nonlimiting embodiment, the second composition is administered daily or every other day until symptoms of the gastrointestinal inflammation are alleviated.

The second composition may be administered by any route providing for delivery of effective amounts of the cannabinoid or NMDA receptor antagonist to a portion of the gastrointestinal tract requiring treatment. Examples of routes of administration include, but are in no way limited to, intravenous, intranasal, oral, topical, transdermal or via inhalation.

As will be understood by the skilled artisan upon reading this disclosure, dosages can be determined by the attending physician, according to the severity and/or extent of the gastrointestinal inflammation to be treated, method of administration, patient's age, weight, contraindications and the like.

In these embodiments, the second composition can be administered before, simultaneously or after administration of the first composition.

In one nonlimiting embodiment the first composition and second composition are combined into a single pharmaceutical formulation administered to the subject.

In one nonlimiting embodiment, an NMDA receptor antagonist is administered with both a CB2 agonist and a FAAH inhibitor to achieve regulation of 2-AG through multiple alternative pathways.

In another nonlimiting embodiment of the present invention, the method for treating gastrointestinal inflammation in a subject suffering therefrom comprises administering to the subject a first composition comprising cannabichromene.

Cannabichromene (CBC) is a cannabinoid found in the Cannabis plant. It bears structural similarity to the other natural cannabinoids, including tetrahydrocannabinol, tetrahydrocannabivarin, cannabidiol, and cannabinol, among others.

In one nonlimiting embodiment, the first composition comprising cannabichromene is administered via a regimen effective to control, reduce or ameliorate inflammation and/or control, reduce or ameliorate any immune response associated with the gastrointestinal condition and/or alleviate symptoms associated with gastrointestinal inflammation in the subject.

In one nonlimiting embodiment, the first composition comprising cannabichromene is administered daily or every other day until symptoms of the gastrointestinal inflammation are alleviated.

The first composition comprising cannabichromene may be administered by any route providing for delivery of effective amounts to a portion of the gastrointestinal tract requiring treatment. Examples of routes of administration include, but are in no way limited to, intravenous, intranasal, oral, topical, transdermal or via inhalation.

As will be understood by the skilled artisan upon reading this disclosure, dosages can be determined by the attending physician, according to the severity and/or extent of the gastrointestinal inflammation to be treated, method of administration, patient's age, weight, contraindications and the like.

Also provided by the present invention are pharmaceutical compositions for treatment of gastrointestinal inflammation. In one nonlimiting embodiment, the pharmaceutical composition comprises a CB2 agonist and an agent which effectively increases an endogenous CB2 agonist or an agent which modifies levels of anandamide (AEA) or 2-arachidonoyl glycerol (2-AG) as well as pharmaceutically acceptable vehicle. In one nonlimiting embodiment, the pharmaceutical composition comprises a CB2 agonist and an inhibitor of fatty acid amide hydrolase (FAAH inhibitor). In another nonlimiting embodiment, the pharmaceutical composition comprises a CB2 agonist, an agent which effectively increases an endogenous CB2 agonist or an agent which modifies levels of anandamide (AEA) or 2-arachidonoyl glycerol (2-AG), and an NMDA receptor antagonist as well as pharmaceutically acceptable vehicle. In one nonlimiting embodiment, the pharmaceutical composition comprises a CB2 agonist, a FAAH inhibitor and an NMDA receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compositions and are physiologically acceptable to a subject. An example of a pharmaceutically acceptable vehicle is buffered normal saline (0.15 M NaCl). The use of such media and agents for pharmaceutically active substances is well known in the art. Except insofar as any conventional medium or agent is incompatible with the therapeutic composition, use thereof in the compositions suitable for pharmaceutical administration is contemplated. Supplementary active compounds can also be incorporated into the compositions.

Dispersions comprising the therapeutic compositions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g. vegetable oil). The proper fluidity can be maintained, for example, by the use of a coating such as lecithin, by the maintenance of the required particle size in the case of dispersion, and by the use of surfactants.

Sterile injectable solutions can be prepared by incorporating the therapeutic compositions in the required amount in an appropriate solvent with one or a combination of ingredients enumerated above, as required, followed by filter sterilization. Generally, dispersions are prepared by incorporating the therapeutic compositions into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compounds are mixed with at least one inert, pharmaceutically acceptable excipient or diluent or assimilable edible vehicle such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compounds in the compositions and preparations may, of course, be varied. The amount of the therapeutic compounds in such therapeutically useful compositions is such that a suitable dosage will be obtained.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The pharmaceutical compositions of therapeutic compounds can also be administered in time-release or depot form, to obtain sustained release of the therapeutic compounds over time. The therapeutic compounds of the invention can also be administered transdermally (e.g., by providing the therapeutic compound, with a suitable vehicle, in patch form).

1. A method for treating gastrointestinal inflammation in a subject suffering therefrom, said method comprising administering to the subject a first composition comprising a CB2 agonist, an agent which effectively increases an endogenous CB2 agonist or an agent which modifies levels of anandamide (AEA) or 2-arachidonoyl glycerol (2-AG) and a second composition comprising a cannabinoid, an N-Methyl-D-aspartate (NMDA) receptor antagonist, or a distinct CB2 agonist, agent which effectively increases an endogenous CB2 agonist or agent which modifies levels of anandamide (AEA) or 2-arachidonoyl glycerol (2-AG).

2. The method of claim 1 wherein the first composition comprises a non-cannabinoid CB2 agonist, a cannabinoid CB2 agonist, an agent which increases levels of AEA, an agent that decreases levels of 2-AG, an inhibitor of fatty acid amide hydrolase or anandamide.

3-7. (canceled)

8. The method of claim 1 wherein the second composition comprises a cannabinoid selected from the group consisting of cannabidiol, tetrahydrocannabinol, cannabichromene, cannabinol, cannabigerol tetrahydrocannabivarin and delta-8-tetrahydrocannabinol.

9. The method of claim 1 wherein the subject is administered a CB2 agonist and an inhibitor of fatty acid amide hydrolase.

10-17. (canceled)

18. The method of claim 1 wherein the second composition comprises a noncompetitive NMDA receptor antagonist.

19. The method of claim 1 wherein the second composition comprises 7-hydroxy-delta6-tetrahydrocannabinol 1,1-dimethylheptyl.

20. The method of claim 1 wherein the subject is administered a CB2 agonist, an inhibitor of fatty acid amide hydrolase and an N-Methyl-D-aspartate (NMDA) receptor antagonist.

21. A method for treating gastrointestinal inflammation in a subject suffering therefrom, said method comprising administering to the subject a first composition comprising cannabichromene.

22. (canceled)

23. The method of claim 1 wherein the subject is suffering from gastritis, esophagitis, colitis, inflammatory bowel disease, Crohn's disease, ulcerative colitis, an immune disorder characterized by gastrointestinal inflammation, or irritable bowel syndrome.

24. A pharmaceutical composition for treatment of gastrointestinal inflammation, said composition comprising a CB2 agonist, an agent which effectively increases an endogenous CB2 agonist or an agent which modifies levels of anandamide (AEA) or 2-arachidonoyl glycerol (2-AG) and a pharmaceutically acceptable vehicle.

25. The pharmaceutical composition of claim 24 wherein the agent which modifies levels of anandamide (AEA) or 2-arachidonoyl glycerol (2-AG) is an inhibitor of fatty acid amide hydrolase.

26. The pharmaceutical composition of claim 24 further comprising an NMDA receptor antagonist.

27. The method of claim 21 wherein the subject is suffering from gastritis, esophagitis, colitis, inflammatory bowel disease, Crohn's disease, ulcerative colitis, an immune disorder characterized by gastrointestinal inflammation, or irritable bowel syndrome.
